(19) United States
(12) Patent Application Publication
(10) Pub. No.: US 2004/0266662 A1
(43) Pub. Date: Dec. 30, 2004

(54) Spheroidal hdl particles with a defined phospholipid composition

(76) Inventors: Kerry-Anne Rye, South Australia (AU); Phillip Barter, South Autralia (AU)

Correspondence Address: R. Neil Sudol/Henry D. Coleman, William J Sapone, Coleman Sudol Sapone, 708 Third Avenue, 14th Floor, New York, NY, 10017-4104, US

(21) Appl. No.: 10/490,497

(22) PCT Filed: Sep. 27, 2002
(86) PCT No.: PCT/AU02/01328

(30) Foreign Application Priority Data
Sep. 28, 2001 (AU) PR 7989

(51) Int. Cl. A61K 38/17; C07K 14/775

(52) U.S. Cl. 514/2; 530/359

(57) ABSTRACT

Spheroidal reconstituted HDL particles with a defined phospholipid composition have been formed for the first time. The spheroidal HDL particle are formed from discoidal HDL particles of defined phospholid composition to which is added in multiple stepwise format unesterified cholesterol carried in a solvent, followed by addition of a LCAT. Lysophopholipids are removed and a specific sequence of addition of reagents are found to be beneficial. Additionally it is found that a ratio of phospholipids to apoprotein A-1 of about 100:1 enhances the result.

This invention relates to a method of preparing spheroidal HDL particles with a defined phospholipid composition, and to lipoprotein particles with a defined phospholipid composition.

### BACKGROUND TO THE INVENTION

One of the roles of the vascular system is the transport of fatty substances such as lipids and cholesterol. These compounds are non-polar and accordingly are not soluble in blood and are generally transported by soluble lipoprotein complexes.

Normal serum contains a number of lipoprotein particles which are separated by ultracentrifugation into large, low density chylomicrons(<1.006 gm/mL;>100 nm), very low density lipoproteins (VLDL, d<1.006 gm/mL; 30-90 nm), intermediate density lipoproteins (IDL, d=1.006-1.019 gm/mL), low density lipoproteins (LDL, d=1.019-1.063 gm/mL; about 20 nm), and high density lipoproteins (HDL, d=1.063-1.21 gm/mL; about 8-12 nm).

The lipoprotein classes can also be identified by means of their associated apolipoproteins. Fourteen major human plasma apolipoproteins have been identified and their associations with lipoproteins characterized. The two major apolipoproteins on HDL are apoA-I and apoA-II. Chylomicrons are associated with apoB₄₈, and apoB₁₀₀ is the predominant apolipoprotein on VLDL and LDL. The apoC proteins are associated with all lipoproteins except LDL. Apolipoprotein E is a constituent of chylomicrons, VLDL, and HDL. Other apolipoproteins, such as Lp(a), apoD and apoF are present in low concentrations.

High density lipoprotein (HDL) transports these materials to the liver for elimination whilst low density lipoprotein (LDL) transports lipid soluble materials to the cells in the body. Normally, these lipoproteins are in balance, ensuring proper delivery and removal of lipid soluble materials. Malfunction of the lipid transport system has been associated with a number of disorders including atherosclerotic and other inflammatory conditions. The study of HDL particles has been an important part of understanding these condition and the lipid transport system, and that study has resulted in a number of suggestions have been made for a therapeutic or preventative approach to these disorders using reconstituted HDL particles.

For HDL the most prominent apolipoprotein components are A-I and A-II which determine the functional characteristics of HDL. Minor amounts of apolipoprotein C-I, C-II, C-III, D, E, J, etc. are embedded in a phospholipid layer and the hydrophobic centre contains cholesterol esters and triglycerides. HDL can exist in a wide variety of different sizes and different mixtures of these constituents depending on the status of remodelling by various plasma factors. In its newly secreted form the HDL particle is composed of apoliprotein with small amounts of associated lipid. Lipid becomes associated with the particle to form a discoidal (pre-beta-migrating HDL). The discoidal particle accepts free cholesterol into its bilayer which is esterified by the action of lecithin:cholesterol acyltransferase (LCAT). The resulting cholesteryl esters move into the center of the discoidal HDL. The HDL particle expands to a spheroidal particle (HDL₃ and HDL₂) as more and more cholesterol is esterified and moved to the center.

It is well established that the phospholipid composition of the high density lipoproteins (HDL)¹ in human plasma is diverse (1,2). While most HDL phospholipids have a choline headgroup, significant amounts of phosphatidylserine, phosphatidylinositol, phosphatidylethanolamine and sphingomyelin are also present in this lipoprotein class (3). The length and unsaturation of HDL phosphatidylcholine sn-2 acyl chains varies widely, with 1-palmitoyl-2-oleoyl phosphatidylcholine (POPC), 1-palmitoyl-2-linoleoyl phosphatidylcholine (PLPC), 1-palmitoyl-2-arachidonyl phosphatidylcholine (PAPC) and 1-palmitoyl-2-docosahexanoyl phosphatidylcholine (PDPC) comprising 12.9, 34.4, 9.1 and 3.6%, respectively, of the total HDL phosphatidylcholines (2).

The impact of this heterogeneity on the structure and function of HDL is poorly understood. For example, it is not known how HDL phospholipid acyl chain length and unsaturation impacts on the ability of plasma factors to remodel HDL from one subpopulation into another. Similarly, it is not known how phospholipid composition affects the dissociation of pre-β-migrating lipid-free or lipid poor apolipoprotein (apo) A-I that occurs during the remodeling of HDL (4,5). The latter issue is of considerable importance because the lipid-free or lipid poor apoA-I that is generated during HDL remodeling may be available for participation in the first step of reverse cholesterol transport (6).

For many years HDL phospholipids were regarded as being important for maintaining structural integrity of HDL, but were thought to have little impact on HDL metabolism (25), and accordingly it was considered that the impact of not having control over the phospholipid composition was not critical. Recent developments however have shown that phospholipid play a larger role. For example, work in this laboratory has established that phospholipid composition influences the ability of HDL to inhibit the cytokine-induced expression of adhesion molecules in human umbilical vein endothelial cells (26). HDL phospholipids also regulate cholesterol efflux from Fu5AH cells (27). Discoidal HDL particles were used in both of these studies.

While those studies have advanced our understanding of how HDL phospholipids impact on HDL function, little is known about they influence HDL structure and their remodeling by plasma factors. These are issues of considerable importance given that HDL phospholipid composition varies widely (2). HDL acquire phospholipids from several sources. These include other lipoproteins, whose phospholipids are transferred to HDL by the activities of phospholipid transfer protein (28) and CETP (29). HDL also acquire phospholipids from chylomicrons that are undergoing lipolysis by lipoprotein lipase (30). These findings highlight the importance of understanding how variations in phospholipid composition regulate the structure, function and remodeling of HDL.

The study of HDL particles has in the past, in large part, been through the use of reconstituted HDL particles where a substantially planar bilayer has the various proteins such as APO AI added thereto. However such discoid rHDL like particles do not mimic and are not a good model for reflecting the behaviour of naturally occurring HDL particles because they do not accurately reflect the shape of HDL particles in vivo. Additionally there are some drawbacks to the use of discoidal rHDL for therapeutic purposes.

A method of preparing spheroidal rHDL particles is in use and involves the addition of unesterified cholesterol from LDL particles to discoidal rHDL like particles and esterification of the cholesterol by use of the enzyme LCAT, which then facilitates the transfer of the cholesterol through the phospholipid layer to the hydrophobic region adjoining the lipid chains of the lipid bilayer. LDL particles are a good source of cholesterol, and because they do not also have APO-AI the LCAT is not as active in esterification of cholesterol to transport into the LDL particle and thus the majority of the LCAT activity is directed to esterification of UC in the HDL particles. The reason for supplying cholesterol in this format is because cholesterol is very difficult to supply in an aqueous form. One problem with the above approach is that at the same time as migration of cholesterol to the HDL particles occurs, there is also concurrent migration of some of the phospholipids. Thus whilst the discoidal rHDL-like particles are able to be provided with a defined phospholipid composition, as soon as the cholesterol is added the balance is upset by the cross migration of LDL derived phospholipids (11).

Until now it has not been possible to investigate issues associated with the influence of specific phospholipid composition in spheroidal rHDL, systematically because of the difficulties associated with obtaining subpopulations of HDL from human plasma that vary only in their phospholipid composition. This problem has been overcome by the present invention by developing a new approach for preparing spherical reconstituted HDL (rHDL) that are comparable in all respects except for their phospholipid composition.

### SUMMARY OF THE INVENTION

The present invention arises from the development of a protocol that results in the formation of spheroidal rHDL whereby cholesterol is delivered in an unesterified form and is incorporated into discoidal rHDL without an associated transfer of phospholipids derived from other sources.

It has been found not possible to add UC (unesterified cholesterol) in the desired quantities together with LCAT to discoidal particles to develop spheroidal rHDL particles. It is believe that results from the manner in which UC is added to the reaction mix and characteristics of LCAT. rHDL particles are suspended in an aqueous medium and UC cannot be added directly to the aqueous medium because UC is hydrophobic. UC is added in a polar solvent such as ethanol, the non formation of spheroidal rHDL particles is believed to be due to a sensitivity of LCAT to solvents such as ethanol, in that the LCAT is degraded by ethanol before the UC can be esterified.

It is found that spheroidal HDL particles can be made by delivery of unesterified cholesterol in a solvent such as ethanol to discoidal HDL like particles in a stepwise process. It is thought that perhaps the collective phospholipid phase of a reaction mix is able to take up the quantity unesterified cholesterol that is supplied so that perhaps the localised concentration of ethanol is less at the phospholipid layer and the adverse effect is reduced at least for a time sufficient for the LCAT to esterify the UC taken up by the phospholipid phase. A sufficient proportion of the UC taken up by the membrane in each step is esterified and transferred to the centre of the rHDL particles so that more unesterified cholesterol can be added and taken into the collective phospholipid phase and the esterification step repeated. The amount of unesterified cholesterol that can be accommodated in the collective phospholipid phase after LCAT action is such that this process is effective in producing spheroidal rHDL. This is found to provide a reliable in vitro method of producing spheroidal HDL particle.

In a first broad form the invention could be said to reside in a method of forming spheroidal reconstituted HDL particles, including the step of forming a disc shaped HDL particle with a defined phospholipid composition, and the step of making the disc shaped HDL particles spheroidal including multiple stepwise addition of unesterified cholesterol carried in a solvent, followed by addition of a cholesterol esterifying agent capable of esterifying the unesterified cholesterol using acyl constituents of the phospholipids. A suitable cholesterol esterifying agent is the enzyme LCAT.

The invention in a second broad aspect might be said to reside in a composition comprising a preparation of spheroidal reconstituted HDL particles (rHDL) with a defined phospholipid composition. The composition of phospholipids might suitably be wholly of one type, or might be a defined mixture of two or more different types.

### BRIEF DESCRIPTION OF THE DRAWINGS

For a better understanding, the invention will now be described with reference to a number of examples which are also represented in the figures wherein,

FIG. 1. Particle size distribution of spherical (POPC)rHDL, (PLPC)rHDL, (PAPC)rHDL and (PDPC)rHDL. Spherical rHDL were prepared as described under “Experimental Procedures”. An aliquot of each sample was subjected to non-denaturing polyacrylamide gradient gel electrophoresis. The profiles represent scans of Coomassie-stained gels. Diameters were calculated by reference to high molecular weight standards of known diameter.

FIG. 2. Packing order of spherical (POPC)rHDL, (PLPC)rHDL, (PAPC)rHDL and (PDPC)rHDL phospholipids. (POPC)rHDL ( ), (PLPC)rHDL ( ), (PAPC)rHDL (◯) and (PDPC)rHDL (●) were labeled with DPH or TMA-DPH. The steady state fluorescence polarization of each sample was determined at 5° C. intervals from 5-45° C. Values represent the mean±sd of at least three determinations. *p<0.001 by ANOVA for (POPC)rHDL vs (PLPC)rHDL, (POPC)rHDL vs (PAPC)rHDL, (POPC)rHDL vs (PDPC)rHDL, (PLPC)rHDL vs (PAPC)rHDL and (PLPC)rHDL vs (PDPC)rHDL. p<0.01 by ANOVA for (PAPC)rHDL vs (PDPC)rHDL.

FIG. 3. Binding of apoA-I-specific monoclonal antibodies to spherical (POPC)rHDL, (PLPC)rHDL, (PAPC)rHDL and (PDPC)rHDL. The association rate constants (ka) (affinity) of six unique apoA-I-specific antibodies for each rHDL were measured by surface plasmon resonance analysis. The apoA-I epitope defined by each antibody is identified by the amino acid residues of the mature protein.

FIG. 4. Unfolding of apoA-I in spherical (POPC)rHDL, (PLPC)rHDL, (PAPC)rHDL and (PDPC)rHDL. (POPC)rHDL, (PLPC)rHDL, (PAPC)rHDL, (PDPC)rHDL and lipid-free apoA-I were incubated with 0-8 M GdnHCl for 0 (◯), 5 ( ), and 24 (●) h. Values represent the mean of at least three determinations. Experimental errors for the wavelength of maximum fluorescence are ±1.0 nm.

FIG. 5. Kinetics of the unfolding of apoA-I in spherical (POPC)rHDL, (PLPC)rHDL, (PAPC)rHDL and (PDPC)rHDL. Spherical (POPC)rHDL ( ), (PLPC)rHDL ( ), (PAPC)rHDL (●), (PDPC)rHDL ( ) and lipid-free apoA-I (◯) were incubated with 3.5 M GdnHCl for 0-24 h. The wavelengths of maximum fluorescence represent the mean±sd of triplicate determinations.

FIG. 6. Incubation of (POPC)rHDL, (PLPC)rHDL, (PAPC)rHDL and (PDPC)rHDL with CETP and a PC/triolein microemulsion: Effect on rHDL composition. The rHDL were mixed with a PC/triolein microemulsion alone or with the microemulsion and CETP. The phospholipid composition of the rHDL and the microemulsions were identical. The final concentration of rHDL CE and microemulsion triolein were 0.1 and 4.0 mM/L, respectively. The samples which did not contain CETP were either maintained at 4° C. or incubated at 37° C. for 24 h. The samples containing CETP (final activity 2.7 units/mL) were incubated at 37° C. for 1, 3, 6, 12 or 24 h. The final volume of the incubation mixtures was 2 mL. When the incubations were complete the rHDL were isolated by ultracentrifugation in the 1.07<d<1.21 g/mL density range. The composition of each sample was determined as described under “Experimental Procedures”. The rHDL CE/apoA-I (●—●) and TG/apoA-I (◯—◯) molar ratios are shown as a function of time. The values represent the mean±s.d. of triplicate determinations.

FIG. 7. Incubation of (POPC)rHDL, (PLPC)rHDL, (PAPC)rHDL and (PDPC)rHDL with CETP and a PC/triolein microemulsion: Effect on rHDL core lipid content. Spherical (POPC)rHDL (●), (PLPC)rHDL (◯), (PAPC)rHDL ( ) and (PDPC)rHDL (Δ) were incubated with a microemulsion and CETP as described in the legend to FIG. 6. The total core lipid concentration of each rHDL preparation is shown as a function of time.

FIG. 8. Incubation of (POPC)rHDL, (PLPC)rHDL, (PAPC)rHDL and (PDPC)rHDL with CETP and a PC/triolein microemulsion: Effect on rHDL particle size. Spherical (POPC)rHDL, (PLPC)rHDL, (PAPC)rHDL and (PDPC)rHDL were incubated with a microemulsion and CETP as described in the legend to FIG. 6, then isolated by ultracentrifugation and subjected to 3/40% non-denaturing gradient gel electrophoresis. Laser densitometric scans of the stained gels are shown.

FIG. 9. Dissociation of apoA-I from (POPC)rHDL, (PLPC)rHDL, (PAPC)rHDL and (PDPC)rHDL following incubation with CETP and a PC/triolein microemulsion. Spherical (POPC)rHDL, (PLPC)rHDL, (PAPC)rHDL and (PDPC)rHDL were incubated with a microemulsion and CETP as described in the legend to FIG. 6. Aliquots of the unprocessed incubation mixtures were electrophoresed on a 3/40% non-denaturing gradient gels and immunoblotted for apoA-I. Scans of the immunoblots are shown. Tracks 1 and 2 show, respectively, rHDL maintained either at 4° C. or incubated at 37° C. for 24 h in the presence of the microemulsion. The rHDL which were incubated with the microemulsion and CETP for 1, 3, 6, 12 and 24 h are shown in Tracks 3, 4, 5, 6, and 7, respectively. Lipid-free apoA-I is shown in Track 8.

FIG. 10. Phospholipid composition of spheroidal particles made from discoidal (POPC)rHDL, (PLPC)rHDL, (PAPC)rHDL and (PDPC)rHDL, are shown in FIGS. 10A, 10B, 10C and 10D respectively.

### DETAILED DESCRIPTION OF THE INVENTION

Various abbreviations are use herein, and include the following:

apoA-I apolipoprotein A-I

BHT butylated hyroxytoluene

BSA bovine serum albumin

CE cholesteryl esters

CETP cholesteryl ester transfer protein

DPH 1,6-diphenyl-1,3,5-hexatriene

GdnHCL guanidine hydrochloride

HDL high density lipoproteins

LCAT lecithin:cholesterol acyltransferase

LDL low density lipoproteins

PC phosphatidylcholine

PAPC 1-palmitoyl-2-arachidonyl phosphatidylcholine

PDPC 1-palmitoyl-2-docosahexanoyl phosphatidylcholine

PLPC 1-palmitoyl-2-linoleoyl phosphatidylcholine

POPC 1-palmitoyl-2-oleoyl phosphatidylcholine

rHDL reconstituted HDL

TBS Tris-buffered saline

TG triglyceride

TMA-DPH 1-(4-trimethylammoniumphenyl)-6-phenyl-1,3,5-hexatriene p-toluenesulfonate

UC unesterified cholesterol

VLDL very low density lipoprotein

Forming spheroidal rHDL particles from discoidal rHDL particles has proven to be a quite difficult task. Presently only one method is known which is the transfer of cholesterol from LDL particles with the help of the enzyme LCAT. The introduction of cholesterol is difficult because the HDL particles are suspended within an aqueous medium, whereas cholesterol is hydrophobic, furthermore LCAT the enzyme responsible for the esterification of cholesterol is found to be solvent sensitive. The approach taken to date has been to introduce LDL particles as the source of cholesterol because the LDL particles can be readily suspended in an aqueous medium. Cholesterol transfers across from the LDL particles to HDL particles, by reason of the action of LCAT which in the presence of APO I on the HDL particles esterifies the cholesterol on the HDL particles only to thereby facilitate internalisation of the HDL particle and progressively “inflate” those particles. However, not only is there exchange of cholesterol, but there is also exchange of phospholipid. A corollary of the phospholipid exchange is that it is not possible to maintain a defined phospholipid composition in spheroidal reconstituted HDL.

The present invention provides for an incremental approach, adding a measured amount of cholesterol in multiple steps. LCAT has in the past been added together with LDL and discoidal rHDL particles, with attendant transfer of phospholipids. Unesterified cholesterol (UC) cannot be added together with LCAT in a single step because UC is hydrophobic and is solvent soluble, whereas LCAT is very sensitive to the presence of solvents and therefore does not remain active long enough to esterify adequate amounts of the UC to provide for spheroidal rHDL particles. A method developed by the inventors provides a means of forming spheroidal rHDL using solvent delivered UC and LCAT. Repeated additions of UC and LCAT provide for an effective method of forming spheroidal rHDL.

The solvent used for carrying the unesterified cholesterol will be a polar solvent that is miscible with water, such as ethanol, however other similar polar solvents may also be used for example isopropanol.

Additionally it is desired to provide for an adsorbing agent that can selectively adsorb the lyso phospholipid that form as a result of the activity of LCAT. This is preferred because the lyso form of phospholipids tend to have detergent activities and thus tend to therefore have adverse effects on the rHDL particles so formed and may have adverse physiological effects should the particles be intended for administration to a patient. The adsorbing agent can be chosen from a range of agents but one preferable such agent is a protein such as albumin, for example bovine serum albumin.

It is also preferred that an LCAT activator is added. A preferred such LCAT activator is β-mercaptoethanol (βME).

The addition of reagents to the reaction mixes for subsequent steps is preferably in the following sequence, lyso phospholipid adsorbing agent, reducing agent, esterification agent, and unesterified cholesterol.

The term spheroidal is one that will be generally understood, and can be confirmed by observation of the particles under an electron microscope. Generally however the molar ratio of esterified cholesterol to apoA-I protein gives a good indication of whether the particle might be considered spheroidal or not. Thus for a ratio of any less that 10:1 is unlikely to be considered spheroidal, whereas a ratio of 15-20:1 is likely to be very much considered spheroidal for a smaller particle size. A ratio of as high as 30-40:1 might be achieved in larger particle sizes.

The formation of disc shaped HDL particles is widely known and can be made using any number of known methods (see for example A. Jonas, Methods in Enzymology 128, 553-582 (1986)). The most frequent lipid used for reconstitution is phosphatidyl choline, extracted either from eggs or soybeans. Other phospholipids may also be used. For reconstitution the lipids are first dissolved in an organic solvent, which is subsequently evaporated under nitrogen. The lipid is bound in a thin film to a glass. A detergent, normally sodium cholate, is added and mixed. The added sodium cholate causes a dispersion of the lipid. After a suitable incubation period the apolipoprotein is added and, the mixture is dialysed to remove the sodium cholate. At the same time lipids and apolipoproteins spontaneously form themselves into reconstituted lipoproteins. As alternatives to dialysis, hydrophobic adsorbents are available which can adsorb detergents (Bio-Beads SM-2, Bio Rad; Amberlite XAD-2, Rohm & Haas) (E. A. Bonomo, J. B. Swaney, J. Lipid Res., 29, 380-384 (1988)), or the detergent can be removed by means of gel chromatography (Sephadex G-25, Pharmacia).

The discoidal rHDL will have a preferred ratio of phospholipids to apoprotein A-I of about 100:1. If this ratio is significantly greater then the discs are somewhat unstable and there a multiple populations with some discs of larger sizes that are not good substrates for LCAT. If the proportion of phospholipid is much lower there is a tendency to not be sufficient phospholipid to allow for esterification of the UC and to permit a stable particle size for spheroidal rHDL.

It may be at the stage of forming the discoidal rHDL that the phospholipid composition is defined so that a defined proportion of certain phospholipids is added or simply one single phospholipid type. Alternatively and perhaps preferably where mixtures of phospholipids are desired it might be preferred to add other lipid to the spheroidal rHDL. This might be achieved by making the spheroidal HDL with a phospholipid composition containing only one type, for example phosphtidyl choline with a defined fatty acid substituent. The second phospholipid might be added to a reaction mixture in the form of a vesicle, and the vesicle might be made wholly of one phospholipid for example phosphtidyl ethanolamine, and the enzyme PLTP might be added for a defined time in a defined concentration. The effect of this is that some of the second phospholipid will be transferred across to the spheroidal rHDL. Certain parameters of the reaction will determine the rate of transfer and may be standardised such that a certain time of transfer will effect a known proportion of phospholipid content in the rHDL particles. The proportion of phospholipids can be checked using known techniques such as mass spectrophotometric analysis of a sample of the spheroidal rHDL particles.

The composition of phospholipids of the spherical rHDL might suitably be wholly of one type, or might be a defined mixture of two or more different types. The phospholipids might be varied accordingly to their head group and such head groups might be chosen from those typically found in significant amounts in HDL, including phosphatidyl serine, phosphatidylinositol, phosphatidyl ethanolamine and sphingomyelin. However other head groups might also be chosen to provide particular characteristics to the spherical rHDL particles and therefore the head groups might additionally be selected from one or more of the groups comprising phosphatidyl glycerol, phosphatidyl serine, phosphatidyl inositol, phsophatidyl ethanolamine, cerebroside or a ganglioside.

It is known that the nature of the head group will vary the characteristic of particles that are formed by them, by general interactions such as bulk or overall charge, and such interactions might be more specific such as the presence of the head groups might be direct triggers or precursors of molecules that have specific physiological effects, or alternatively may influence directly the interaction with effector cells or receptor molecules.

In the alternative or additionally the fatty acyl substituents might be varied. The most common fatty acid substituents of phospholipids of HDL particles includes 1-palmitoyl-2-oleoyl-, 1-palmitoyl-2-linoleoyl-, 1-palmitoly-2-arachadonyl-, 1-palmitoyl-2-docosahexanoyl. However other fatty acyl groups might also be chosen to provide particular characteristics to the spherical rHDL particles and therefore fatty acyl groups might be selected from those having acyl chains of about 12 to about 18 carbon atoms.

Exemplary phospholipids that might be useful in the invention include, phosphatidylcholine, phosphatidylglycerol, β, γ-dipalmitoyl-α-lecithin, sphingomyelin, phosphatidylserine, phosphatidic acid, phosphatidylethanolamine, phosphatidylinositol, cephalin, cardiolipin, cerebrosides, dicetylphosphate, dioleoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phospatidylcholine, dimyristoylphosphatidylcholine, 1-myristoyl-2-palmitoylphosphatidylcholine, 1-palmitoyl-2-myristoylphosphatidylcholine, 1-palmitoyl-2-stearoylphosphatidylcholine, dioleophosphatidylethanolamine, dilauroylphosphatidylcholine, sphingolipids, diphosphatidylglycerol, dimyristoylphosphatidylglycerol, dipalmitoylphosphatidylglycerol, distearoylphosphatidylglycerol, dimyristoylphosphatidic acid, dipalmitoylphosphatidic acid, dimyristoylphosphatidylethanolamine, dipalmitoylphosphatidylserine, brain phosphatidylserine, brain sphingomyelin, dipalmitoylsphingomyelin, distearoylsphingomyelin, galactocerebroside, gangliosides, dilaurylphosphatidylcholine, (1,3)-D-mannosyl-(1,3)diglyceride, aminophenylglycoside and 3-cholesteryl-6′-(glycosylthio)hexyl ether glycolipids. and the like.

Non-phosphorus containing lipids may also be used in the rHDL of present invention. These include, e.g., stearylamine, docecylamine, acetyl palmitate, fatty acid amides, and the like. Additional lipids suitable for use in the liposomes of the present invention are well known to persons of skill in the art and are cited in a variety of well known sources, e.g., McCutcheon's Detergents and Emulsifiers and McCutcheon's Functional Materials, Allured Publishing Co., Ridgewood, N.J., both of which are incorporated herein by reference.

A particularly preferred defined spheroidal rHDL particle has only PLPC as the phosholipid content. PLPC is a good substrate for LCAT to work on and additionally it is thought to have a role in inhibition adhesion giving it anti-inflammatory properties. This may provide for benefits in prevention of atherosclerosis.

The defined composition of phospholipids might be a range of combinations of one or more applicable phospholipids.

The spherical rHDL particles may also be varied with regards to the protein constituent. Thus the HDL particle will necessarily include apo-AI proteins but other HDL proteins may also be present and these might include preferably at least additionally apoA-II and perhaps also apoA-IV, apoC and apoE. Other proteins may also be added depending on what the spheroidal rHDL particle is intend for. Thus therapeutically active agents might be carried on the phospholipid layer and these maybe protein, glycoprotein or lipoproteins. The protein of these might be chimeric protein with a portion that interacts with the defined phospholipid composition in a desired manner, thus for example these may have a defined rate of release.

The apoprotein might be used in this invention might be purified from lipoprotein particles isolated from human or animals. Purification of apoproteins from lipoproteins is a well known and are number of protocols are known (34-43). Alternatively the apoproteins might be formed recombinantly. Several apoproteins have been cloned in microorganisms and sequenced and such overexpressed apoproteins might be used. Chimeric recombinant apoproteins might also be used such chimeric proteins comprising domains from various apoproteins combined, or combinations of apoprotein domains and domains that might provide therapeutic benefit.

Lipid protective agents, such as BHT may also be included in the lipids forming the liposomes, to protect the lipid components against free radical damage

Several lines of evidence based on data obtained in vivo implicate the HDL and its major protein component, ApoA-I, in the prevention of atherosclerotic lesions, and potentially, the regression of plaques making these attractive targets for therapeutic intervention. First, an inverse correlation exists between serum ApoA-I (HDL) concentration and atherogenesis in man (Gordon & Rifkind, 1989, N. Eng. J. Med. 321:1311-1316; Gordon et al., 1989, Circulation 79:8-15). Indeed, specific subpopulations of HDL have been associated with a reduced risk for atherosclerosis in humans (Miller, 1987, Amer. Heart 113:589-597; Cheung et al., 1991, Lipid Res. 32:383-394); Fruchart & Ailhaud, 1992, Clin. Chem. 38:79).

Lipoprotein particles have interaction with a range of compound in addition to lipids and these may suggest other areas of potential therapeutic benefit. Some such interactions include those with individual components of the complement system, and can therefore influence their activity; components of the coagulation systems are likewise known which are found to be associated with certain lipoproteins; acute phase proteins such as serum amyloid A (SAA) are found in the HDL fraction; and furthermore the adsorption of certain proteins on surfaces can be influenced by pretreatment with lipoproteins.

Lipoproteins can also influence cellular activity by reason of alteration in specific or non-specific binding to cells. The platelet activation can be inhibited by binding of HDL or stimulated by addition of LDL. Monocytes and macrophages have receptors for lipoproteins; the binding or uptake of lipoproteins can lead to changes in the activities of these cells as well as for neutrophils. The growth of tumor cells, shown using glyoblastoma cells as an example, can also be influenced through lipoproteins.

There are also reports that describe an antimicrobial activity for lipoproteins. Thus viruses can have been inactivated by means of lipoproteins, or, using trypanosomes as an example, parasites can be influenced or respectively inhibited.

It might be desired to use these rHDL as a therapeutic delivery vehicle thus having a predetermined proportion of phospholipids having for example a longer chain length for a predetermined release of a therapeutic. Is it possible that HDL might be a vehicle for the targeted delivery of therapeutics. The degree of exposure of apo AI in HDL might be modulated.

It is therefore anticipated that HDL particles of the present invention may be utilised therapeutically. Thus they might be administered to a patient for treatment, for example parenterally as a pharmaceutically acceptable preparations. Alternatively HDL particles of the present invention might be used in ex vivo treatments for example coupled to a solid support for filtering a body fluid for example blood.

### EXAMPLE 1

Four types of spherical rHDL were used for this study. The rHDL contained either POPC, PLPC, PAPC or PDPC as the sole phospholipid constituent. The rHDL preparations all contained cholesteryl esters (CE) as their only core lipid and apoA-I as the only apoliprotein. The preparations were all similar in size and had comparable lipid/protein ratios. In other words, the only difference of note between the four rHDL preparations was the length and unsaturation of their phosphatidylcholine sn-2 acyl chains.

Experimental Procedures

Preparation of Lipid-Free ApoA-I

HDL were isolated from autologously donated samples of pooled human plasma (Gribbles Pathology, Adelaide, South Australia) by ultracentrifugation in the 1.07<d<1.21 g/mL density range (7). The isolated HDL were delipidated by standard techniques (8) and the resulting apoHDL was subjected to anion exchange chromatography on a Q-Sepharose Fast Flow column (Pharmacia, Uppsala, Sweden) attached to an FPLC system (Pharmacia) (9,10). This procedure was carried out at room temperature. ApoA-I appeared as a single band following electrophoresis on a 20% homogeneous SDS gel (Phast System, Pharmacia) and Coomassie staining.

Preparation of Lecithin:Cholesterol Acyltransferase (LCAT)

LCAT was prepared as described (11) and concentrated 10-fold by ultrafiltration (Amicon, Danvers, Mass.). LCAT activity was assessed using POPC/unesterified cholesterol (UC)/apoA-I discoidal rHDL labeled with [1α,2α(n)-³H]cholesterol ([³H]UC) (Amersham Pharmacia Biotech) as the substrate (12). The POPC was obtained from Avanti Polar Lipids (Alabaster, Ala.). The UC was from Sigma. The activity was linear when less than 30% of the [³H]UC was esterified. The LCAT preparations used in this study generated 1.0-2.4 μmol CE/mL LCAT/h.

Preparation of CETP

CETP was prepared as described (4). The activity of the preparations was determined as the transfer of [³H]CE from [³H]CE-HDL₃ to low density lipoproteins (LDL) (13,14). The LDL were isolated by sequential ultracentrifugation in the 1.019<d<1.055 g/mL density range. The activity was linear as long as less than 30% of the [³H]CE transferred from HDL₃ to LDL. Activity is expressed in units/mL, with 1 unit being the transfer activity of 1 mL of a preparation of pooled, human lipoprotein-deficient plasma. The activities of the CETP preparations used in this study varied from 21.2 to 39.0 units of activity/mL.

Preparation of Spherical rHDL.

Discoidal rHDL containing UC, apoA-I and either POPC, PLPC, PAPC or PDPC (Avanti), were prepared by the cholate dialysis method (15). The starting phosphatidylcholine (PC)/UC/apoA-I molar ratio was 110:5:1. The discoidal rHDL were dialysed against 5×1 L Tris-buffered saline (TBS) (10 mM Tris, 150 mM NaCl), pH 7.4, containing 50 μM diethylenetriamine pentaacetic acid, 0.006% (w/v) and 10 μM butylated hydroxytoluene (BHT). Chelex 100 resin (Bio-Rad, Hercules, Calif.) (2 g/L) was added to the TBS to prevent inadvertent oxidation.

The discoidal rHDL were converted into spherical rHDL by sequentially adding unesterified cholesterol dissolved in ethanol to incubations of the rHDL and LCAT. In a typical incubation for the preparation of spherical (POPC)rHDL, discoidal (POPC)rHDL (final UC concentration 0.2 mM/L) were mixed with bovine serum albumin (BSA) (final concentration 40 mg/mL) and β-mercaptoethanol (final concentration 4 mM/L), then incubated at 37° C. for 30 min with LCAT (2.6 mL of a preparation that esterified 1.1 μmol CE/mL LCAT/h). The final volume of the incubation mixture was 10.1 mL.

At 30 min, 1.2 μmol of UC (0.052 mL of 24.6 mM/L UC in ethanol) and an additional 0.65 mL of LCAT were added to the incubation mixture. Extra BSA and β-mercaptoethanol were also added so that the final concentrations of both constituents were maintained at 40 mg/mL and 4 mM/L, respectively. The additions of UC, LCAT, BSA and β-mercaptoethanol were repeated at 30 minute intervals until 7 h had elapsed. The final volume of the incubation mixture was 23.4 mL. The incubation was then continued without further additions until the total incubation time was 24 h.

The resulting spherical (POPC)rHDL were isolated by ultracentrifugation at 4° C. in the 1.07<d<1.21 g/mL density range, with a single spin at the upper density and two spins at the lower density. Density adjustments were made with solid KBr. The first spin at 1.07 g/mL was carried out for 16 h at 55,000 rpm in a 55.2 Ti rotor in a Beckman L8-M ultracentrifuge. The 1.21 g/mL spins were carried out for 16 h at 100,000 rpm using a 100.4 Ti in a Beckman TLA-100 Tabletop ultracentrifuge. The spherical (POPC)rHDL were dialysed against 3×1 L TBS before use.

The amount of LCAT required to convert discoidal rHDL into spherical rHDL varied according to the phospholipid composition of the starting discoidal rHDL. This reflects variations in the substrate specificity of LCAT (16). For example, while a total volume of 11.8 mL of LCAT was required to convert discoidal (POPC)rHDL into spherical (POPC)rHDL, the conversion of an equivalent amount of discoidal (PLPC)rHDL into spherical (PLPC)rHDL required 10.0 mL of the same preparation of LCAT. In the case of (PAPC)rHDL, 21.7 mL of the same LCAT was required to convert discs into spheres, while 42.2 mL of LCAT was required to obtain spherical (PDPC)rHDL.

Preparation of Microemulsions

Phospholipid/triolein microemulsions were prepared as described by Martins et al. using triolein and either POPC, PLPC, PAPC or PDPC. The starting triolein/phospholipid molar ratio was 2.5/1.0 (17).

Incubations

All incubations were carried out in stoppered plastic tubes in a shaking water bath maintained at 37° C. Non-incubated control samples were stored at 4° C. Details of the individual incubations are described in the figure legends.

Determination of rHDL CE Composition

The content of rHDL CE was determined by HPLC with UV_{210 nm} detection as described previously (18). Briefly, the rHDL were extracted with methanol/hexane, the organic phase separated, dried and then resuspended in methanol/tert.butylalcohol (1/1, v/v). Lipids were separated by reverse-phase HPLC and individual CE quantified at 210 nm by area comparison with authentic standards (Sigma).

Spectroscopic Studies

A Perkin-Elmer LS-50 luminescence spectrometer was used for these studies. Intrinsic fluorescence emission spectra were recorded from 300-380 nm using an excitation wavelength of 295 nm and excitation and emission band passes of 5 and 6 nm, respectively. The same excitation wavelength was used to obtain intrinsic fluorescence polarization data.

The unfolding of apoA-I was determined by incubating the rHDL preparations at 25° C. for 0-24 h in the presence of 0-8.0 M guanidine hydrochloride (11).

Phospholipid acyl chain and headgroup packing order was assessed as the steady-state fluorescence polarization of rHDL labeled respectively with 1,6-diphenyl-1,3,5-hexatriene (DPH) and 1-(4-trimethylammoniumphenyl)-6-phenyl-1,3,5-hexatriene p-toluenesulfonate (TMA-DPH) (7). Polarization measurements were made at 5° C. intervals from 5 to 45° C. The phospholipid/probe molar ratio was 500/1. The labeling procedures are described elsewhere (7).

Surface Plasmon Resonance Analysis

A Biacore 2000 biosensor (Pharmacia) was used to measure the ka (association rate constant) of each rHDL for six unique apoA-I-specific monoclonal antibodies. Maximum amounts of rabbit anti-mouse Fc (RAM-Fc) were immobilized on all four flowcells of a CM5 chip using amine coupling as described (19). Each rHDL (analyte) was then bound by the captured antibodies.

Data was collected at a high data collection rate (10 Hz). Evaluation of the data began with synchronizing the injection times and zeroing each sensorgram to baseline. For each antibody and rHDL combination, a set of sensorgrams was evaluated with a 1:1 (Langmuir) association (k observed) model. The ka was obtained from the slope of the plot of k observed v's analyte concentration. Microsoft Excel 2000 was used for statistical analysis. The t-test for two-tailed distribution and two-sample unequal variance (heteroscedastic) was used to determine significant differences.

Following covalent attachment of the RAMFc, the individual purified antibodies were injected at protein concentrations between 1.9 and 10 μg/mL to give approximately 400 response units.

Mass Spectroscopy

Mass spectra were acquired using an API-100 ion spray mass spectrometer (PE/Sciex) using an ion source voltage equal to 5,000V and an orifice voltage equal to 70V. Data were collected at 0.1 amu resolution over a mass/charge (m/z) range of 100-1,000.

Immunoblotting

Aliquots of unprocessed incubation mixtures were electrophoresed on 3/40% non-denaturing polyacrylamide gradient gels and transferred electrophoretically to nitrocellulose membranes (5). ApoA-I was detected by enhanced chemiluminescence (Amersham Life Sciences, Inc).

Other Techniques

Spherical rHDL surface charge was determined by agarose gel electrophoresis (20). Non-denaturing 3/40% polyacrylamide gradient gel electrophoresis was used to determine rHDL diameters. The gradient gels were prepared according to the method of Rainwater et al. (21). A Cobas Fara automated analyzer (Roche Diagnostics, Zurich, Switzerland) was used for all chemical analyses. ApoA-I concentrations were determined by the method of Lowry et al. (22), using BSA as a standard. Enzymatic kits (Boehringer Mannheim GmbH, Germany) were used to measure phospholipid, UC, and total cholesterol concentrations. CE concentrations were calculated as the difference between the total cholesterol and UC concentrations. The rHDL preparations were cross linked according to the method of Staros (23).

Statistical Analysis

Unless stated otherwise the data analysis package in Microsoft Excel 98 was used for statistical analyses. The t-test for paired samples was used to determine whether differences between values were significant. ANOVA: Two-factor with repeated measures was used to assess differences between data sets. In all cases significance was determined as p<0.05.

Results

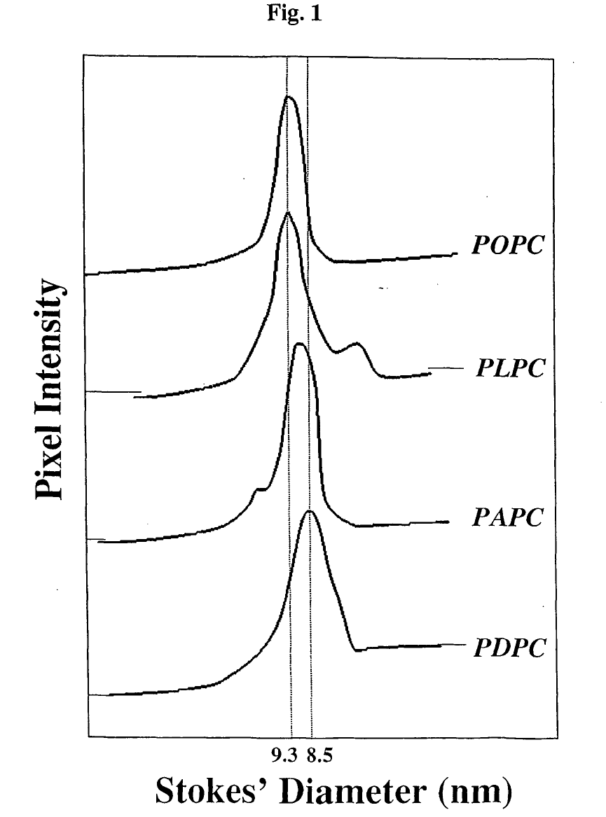

Physical Properties of (POPC)rHDL, (PLPC)rHDL, (PAPC)rHDL and (PDPC)rHDL (FIG. 1, Tables I-II).

Spherical rHDL containing either POPC, PLPC, PAPC or PDPC as the only phospholipid constituent were prepared as described under “Experimental Procedures”. As judged by non-denaturing gradient gel electrophoresis the (POPC)rHDL and (PLPC)rHDL were 9.3 nm in diameter. The respective diameters of the (PAPC)rHDL and (PDPC)rHDL were 9.0 and 8.5 nm (FIG. 1). The (PLPC)rHDL also contained a minor population of smaller particles, 7.8 nm in diameter.

The physicochemical properties of the rHDL preparations are shown in Table I. The CE/apoA-I molar ratios ranged from 22.9/1.0, for the (PDPC)rHDL, to 24.4/1.0 for the (PAPC)rHDL. There was some variation in the phospholipid/apoA-I molar ratios, which ranged from 17.4/1.0 for the (PAPC)rHDL to 26.5/1.0 for the (PLPC)rHDL. As judged by covalent, chemical cross linking, all of the rHDL preparations contained three apoA-I molecules/particle.

Mass spectroscopic analysis confirmed that the phospholipid composition of the spherical rHDL preparations was identical to the phospholipid composition of the starting discoidal rHDL. Lysophosphatidylcholine was not detected in any of the spherical rHDL preparations.

Agarose gel electrophoresis was used to assess the electrophoretic mobilities of the rHDL (Table I). All of the preparations were more negatively charged than lipid-free apoA-I. The (POPC)rHDL and (PLPC)rHDL surface charge was comparable to that of plasma HDL. The (PAPC)rHDL and (PDPC)rHDL, by contrast, were more negatively charged than either (POPC)rHDL, (PLPC)rHDL or plasma HDL.

The local rotational motions of the rHDL Trp residues were assessed by intrinsic fluorescence polarization (Table I). The rHDL polarization values were all decreased relative to lipid-free apoA-I. This confirms earlier reports from this laboratory showing that lipid-association increases the rotational freedom of apoA-I Trp residues (4). The intrinsic polarization values for the (PAPC)rHDL and (PDPC)rHDL were significantly lower than the values obtained for the (POPC)rHDL and (PLPC)rHDL. This suggests that the rotational motion of rHDL apoA-I Trp residues increases with increasing length and/or unsaturation of the rHDL sn-2 phospholipid acyl chains.

Fluorescence spectroscopy was used to compare the environments of the rHDL apoA-I Trp residues. The wavelengths of maximum fluorescence of the (POPC)rHDL, (PLPC)rHDL and (PAPC)rHDL were blue shifted relative to that of lipid-free apoA-I (Table I). This indicates that the apoA-I Trp residues in (POPC)rHDL, (PLPC)rHDL and (PAPC)rHDL are in a more hydrophobic environment than the Trp residues in lipid-free apoA-I. The wavelength of maximum fluorescence of the apoA-I in (PDPC)rHDL was, by contrast, comparable to that of lipid-free apoA-I. When these results are taken together with the intrinsic polarization data it suggests that the conformation apoA-I varies in the rHDL preparations.

LCAT generates CE by hydrolysing HDL phospholipid sn-2 acyl ester bonds (1). The resulting non esterified fatty acids transacylate the 3-hydroxyl group of cholesterol. LCAT has also been reported to hydrolyse HDL phospholipid sn-1 acyl ester bonds. This is particularly evident in HDL that contain phospholipids with long, polyunsaturated sn-2 acyl chains (1). HPLC was used to determine whether LCAT hydrolysed phospholipid sn-1 acyl ester bonds in the present study (Table II). In the case of (POPC)rHDL, cholesteryl oleate and cholesteryl palmitate comprised 88.7 and 10.2% of the total rHDL CE, respectively. Similar results were obtained for the (PLPC)rHDL, with cholesteryl linoleate and cholesteryl palmitate respectively comprising 87.2 and 11.0% of the total CE. These results confirm that LCAT preferentially hydrolyses sn-2 acyl ester bonds in (POPC)rHDL and (PLPC)rHDL. In the case of (PAPC)rHDL and (PDPC)rHDL there was increased hydrolysis of sn-1 acyl ester bonds by LCAT, with cholesteryl palmitate comprising 23.0 and 20.5% of the total CE in the (PAPC)rHDL and (PDPC)rHDL, respectively.

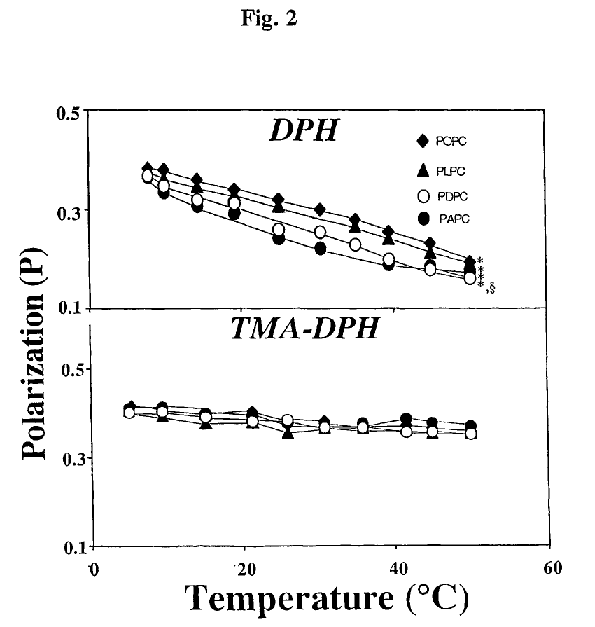

Structural Properties of (POPC)rHDL, (PLPC)rHDL, (PAPC)rHDL and (PDPC)rHDL. (FIGS. 2-5, Table III).

To determine how variations in sn-2 acyl chain length and unsaturation affect rHDL phosphatidylcholine acyl chain and headgroup packing order, the preparations were labeled with either DPH or TMA-DPH. The steady state fluorescence polarization of the samples was determined as described under “Experimental Procedures” (FIG. 2).

As expected, the polarization values of the rHDL labeled with DPH decreased with increasing temperature (FIG. 2). The polarization of these samples also decreased with increasing length and unsaturation of the rHDL phosphatidylcholine sn-2 acyl chains. In general, the polarization values for (POPC)rHDL (closed diamonds)>(PLPC)rHDL (closed triangles)>(PAPC)rHDL (open circles)>(PDPC)rHDL (closed circles). As judged by ANOVA, the differences between the preparations were statistically significant with p<0.001 for (POPC)rHDL v's (PLPC)rHDL, (POPC)rHDL v's (PAPC)rHDL, (POPC)rHDL v's (PDPC)rHDL, (PLPC)rHDL v's (PAPC)rHDL and (PLPC)rHDL versus (PDPC)rHDL (*). In the case of (PAPC)rHDL versus (PDPC)rHDL, p<0.01 ( ).

The polarization of the TMA-DPH-labeled rHDL also decreased with increasing temperature. There were, however, no significant differences between the rHDL preparations, indicating that phospholipid acyl chain length and unsaturation has no effect on rHDL phospholipid head group packing order.

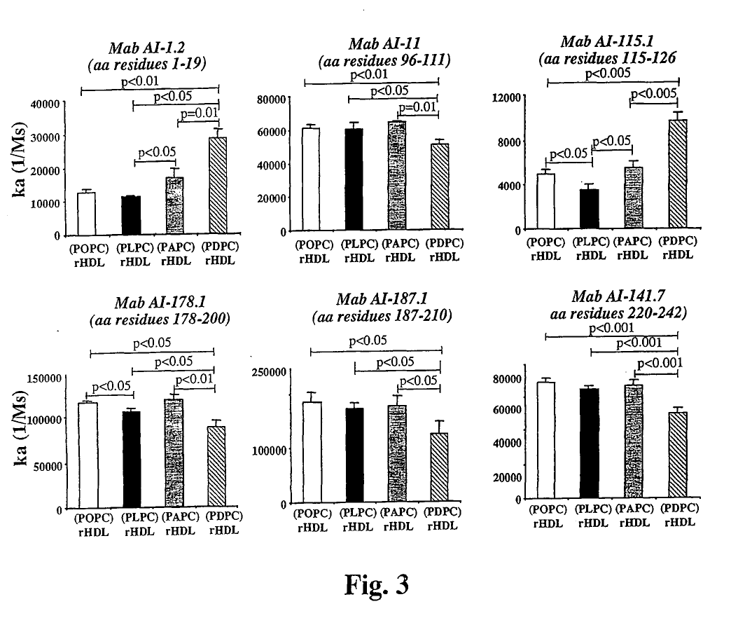

Surface plasmon resonance was used to determine how the length and unsaturation of rHDL sn-2 phosphatidylcholine acyl chains affects the conformation of apoA-I (FIG. 3). Six unique epitopes between the N— and the C-terminal regions of apoA-I were tested. The association rate constants (ka) of a given antibody for each of the four rHDL preparations were calculated. The immediate N-terminal epitope of apoA-I, consisting of aa residues 1-19, had a higher affinity for its antibody on (PDPC)rHDL compared with all of the other rHDL. This suggests that the N-terminal region of apoA-I is more accessible on (PDPC)rHDL compared with the other rHDL. In contrast, compared with all other rHDL the three C-terminal epitopes that were examined had a significantly less affinity for their respective antibodies. These epitopes were defined by the aa residues 178-200, the epitope at aa residues 187-210 and the immediate C-terminal epitope comprising residues 220-242. This suggests that the N-terminal region of the apoA-I in (PDPC)rHDL is more exposed than the C-terminal region of (POPC)rHDL. Interestingly, the affinity of two adjoining regions of apoA-I (epitopes 96-111 and 115-126) changed in opposite directions. Finally, there were few significant differences observed among the (POPC)rHDL, (PLPC)rHDL, and (PAPC)rHDL throughout apoA-I, with the greatest differences observed at epitope 115-126 which represents a proline-punctuated β-turn between adjoining α-helices.

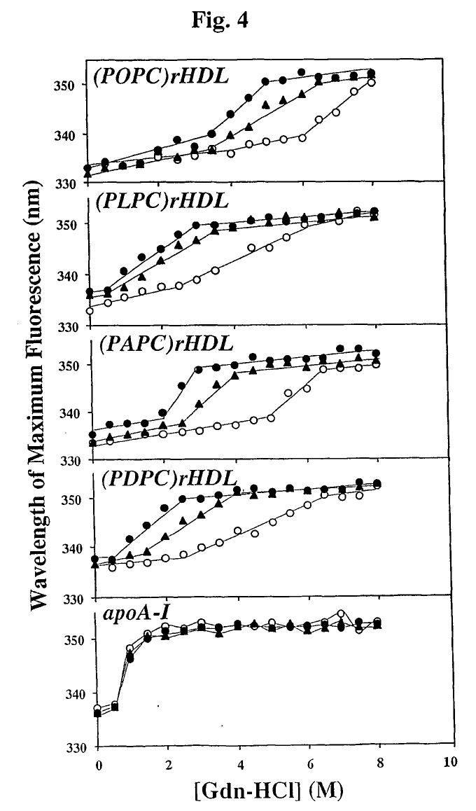

The results of the surface plasmon resonance studies showing that the conformation of apoA-I varied in the rHDL preparations suggested that phospholipid acyl chain composition may regulate the penetration of apoA-I into the rHDL surface. This, in turn, could affect the stability of the apoA-I and its ability to dissociate from the rHDL surface. These issues were addressed by determining the wavelength of maximum fluorescence of each rHDL preparation after incubation with varying concentrations of GdnHCl (FIG. 4). The results at 0 h, immediately after addition of GdnHCl to the samples, (open circles), as well as the results after 5 h (closed triangles) and 24 h (closed circles) of incubation are shown. The unfolding of lipid-free apoA-I is shown in the lower panel. As reported previously, lipid-free apoA-I unfolded rapidly and completely when incubated in the presence of GdnHCl (11). The unfolding of the lipid-associated apoA-I was, by contrast, much slower and time dependent.

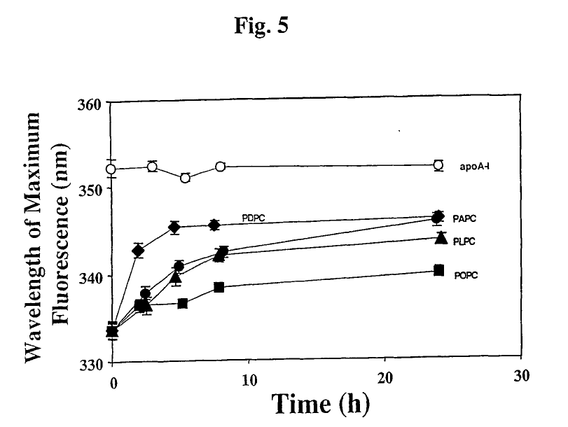

The kinetics of the unfolding of apoA-I was determined by incubating the rHDL preparations for 0-24 h with 3.5 M GdnHCl (FIG. 5). As the wavelengths of maximum fluorescence of the rHDL preparations varied at T=0 h (Table I), the values were normalized to 333.7 nm at this time point. The lipid-free apoA-I unfolded rapidly and completely (open circles). The lipid-associated apoA-I, by contrast, unfolded at a slower rate, with (PDPC)rHDL (closed diamonds)>(PLPC)rHDL (closed triangles)˜(PAPC)rHDL (closed circles)>(POPC)rHDL (closed squares).

The concentration of GdnHCl required to achieve 50% unfolding of apoA-I was also determined (Table III). As reported elsewhere, lipid-free apoA-I was unfolded by 50% at 1.0 M GdnHCl (11). A concentration of 3.6-3.8 M GdnHCl was required to achieve 50% unfolding of (POPC)rHDL. Lower concentrations of GdnHCl (1.7-2.0 M) were required to achieve 50% unfolding of the apoA-I in the (PLPC)rHDL, (PAPC)rHDL and (PDPC)rHDL. These results are consistent with the higher free energy of unfolding (ΔG_{H}_{₂}_{O}⁰) for (POPC)rHDL compared to (PLPC)rHDL, (PAPC)rHDL and (PDPC)rHDL. These differences cannot be attributed to variations in the number of GdnHCl binding sites (Δn) in the apoA-I.

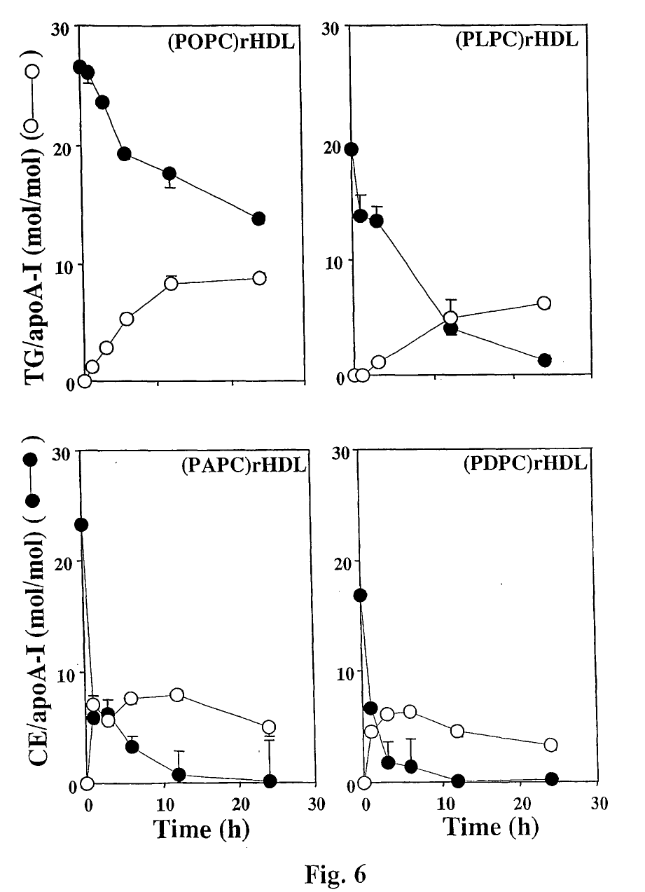

Remodeling of rHDL by CETP (FIGS. 6-9, Table IV).

Earlier work from this laboratory has shown that when HDL are incubated with CETP and triglyceride (TG)-rich particles such as VLDL or Intralipid®, CE and TG transfer between the HDL and the TG-rich particles (7). These transfers are associated with the remodeling of the HDL into smaller particles and may be accompanied by the dissociation of lipid-free, or lipid-poor, apoA-I (4,5).

To determine how HDL phospholipid composition affects these processes, (POPC)rHDL, (PLPC)rHDL, (PAPC)rHDL and (PDPC)rHDL were incubated with CETP and phospholipid/triolein microemulsions. As the likelihood of CETP transferring phospholipids between the microemulsions and the rHDL was high (24), the microemulsions were prepared with either POPC, PLPC, PAPC or PDPC as the only phospholipid. Thus, the (POPC)rHDL were incubated with a POPC/triolein microemulsion. Likewise, the (PLPC)rHDL, (PAPC)rHDL and (PDPC)rHDL were incubated with microemulsions containing triolein and either PLPC, PAPC or PDPC.

This ensured that the phospholipid composition of the rHDL did not change during the incubations with CETP.

The composition of the rHDL at each time point is shown in Table IV. The stoichiometry of the (POPC)rHDL changed minimally when they were incubated for 24 h at 37° C. with the POPC/triolein microemulsion in the absence of CETP. The PC/apoA-I molar ratio of the (PLPC)rHDL, (PAPC)rHDL and (PDPC)rHDL by contrast, increased under these conditions. This indicates that phospholipids transferred spontaneously from the microemulsions to the (PLPC)rHDL, (PAPC)rHDL and (PDPC)rHDL during incubation at 37° C. in the absence of CETP.

When CETP was included in the incubations, the CE/apoA-I molar ratio of the rHDL decreased (closed circles) and there was a concomitant increase in the TG/apoA-I molar ratio (open circles) (Table IV, FIG. 6). Transfers of CE and TG between the (POPC)rHDL and the POPC/triolein microemulsion were slow compared to the other rHDL preparations. After 24 h of incubation with CETP, CE was still the predominant core lipid in the (POPC)rHDL with the respective CE/apoA-I and TG/apoA-I molar ratios being 13.8/1.0 and 8.8/1.0 (Table IV).

This was not the case for the (PLPC)rHDL. After 12 h of incubation with CETP and the PLPC/triolein microemulsion, the CE/apoA-I and TG/apoA-I molar ratios of the (PLPC)rHDL were approximately equal, while at 24 h they were 1.2/1.0 and 6.2/1.0, respectively (Table IV, FIG. 6). Transfers of CE and TG between the microemulsions and the (PAPC)rHDL and (PDPC)rHDL were rapid relative to the other rHDL preparations. Both the (PAPC)rHDL and (PDPC)rHDL were completely depleted of CE after 12 h of incubation in the presence of CETP.

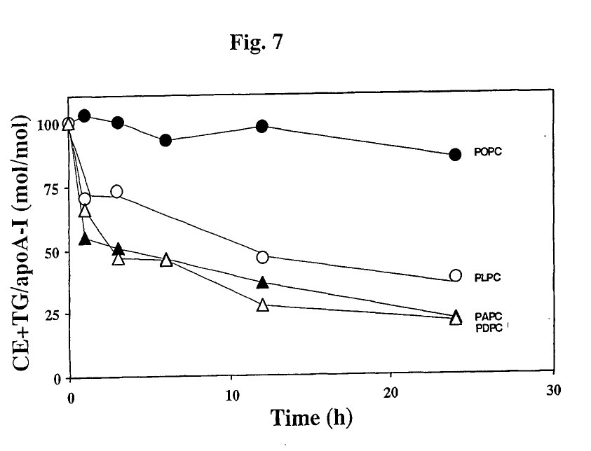

Previous work from this laboratory has shown that the core lipid content of rHDL decreases when they are incubated with CETP and triglyceride-rich particles (either Intralipid® or VLDL) (4). To ascertain how HDL phospholipid composition affects this reduction in core lipid content, the rHDL CE+TG/apoA-I molar ratios were calculated from the data in Table IV, normalized to 100% at 0 h, and plotted as a function of time (FIG. 7). After 24 h of incubation with CETP and a POPC/triolein microemulsion, the CE+TG/apoA-I molar ratio of the (POPC)rHDL had decreased by 15% (closed circles). The core lipid content of the (PLPC)rHDL, by contrast, decreased by 62% during this time (open circles), while that of the (PAPC)rHDL (closed triangles) and (PDPC)rHDL decreased by 79%.

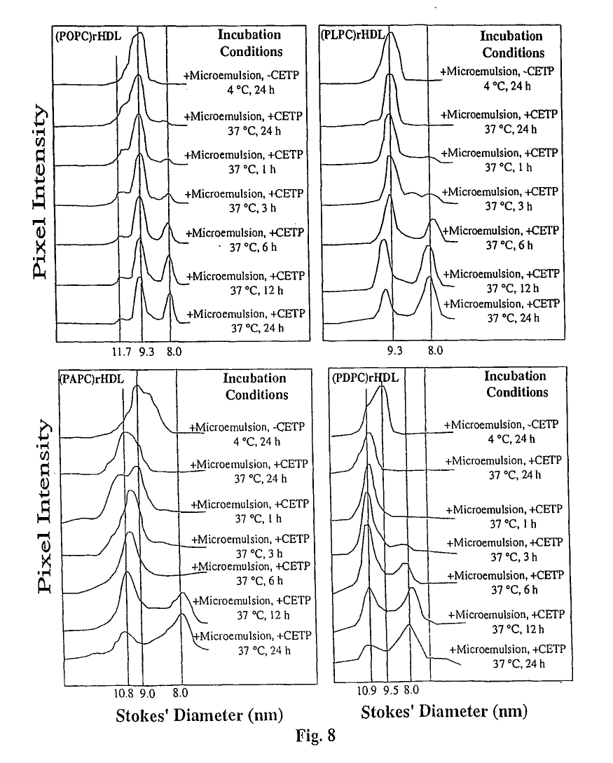

To determine whether the loss of core lipids was associated with a reduction in rHDL size, the samples were also subjected to non denaturing gradient gel electrophoresis (FIG. 8). The (POPC)rHDL diameter was not affected by incubation for 24 h in the absence of CETP. After 1 h of incubation with CETP a minor population of small particles, 8.0 nm in diameter appeared. As the incubation proceeded the (POPC)rHDL were progressively converted into smaller particles. By 24 h, 38% of the (POPC)rHDL were 8.0 nm in diameter.

Although the (PLPC)rHDL acquired additional phospholipids when they were incubated with the PLPC/triolein microemulsion in the absence of CETP (Table IV), their size was not affected (FIG. 8). When CETP was present in the incubation there was a progressive appearance of small particles 8.0 nm in diameter. After 24 h of incubation with CETP, 70% of the (PLPC)rHDL were converted into particles 8.0 nm in diameter. This result is consistent with the data in FIG. 7, which shows that the reduction in (PLPC)rHDL core lipid content is enhanced relative to (POPC)rHDL.

When the (PAPC)rHDL were incubated for 24 h with a PAPC/triolein microemulsion in the absence of CETP the PC/apoA-I molar ratio increased from 15.1/1.0 to 33.7/1.0 (Table IV). This was accompanied by an increase in particle size from 9.0 to 10.8 nm. Similar changes in the PC/apoA-I molar ratio and particle size were apparent when the (PDPC)rHDL were incubated with the PDPC/triolein microemulsion. After 24 h of incubation in the presence of CETP, approximately 65% of the (PAPC)rHDL and 78% of the (PDPC)rHDL were converted to 8.0 nm particles

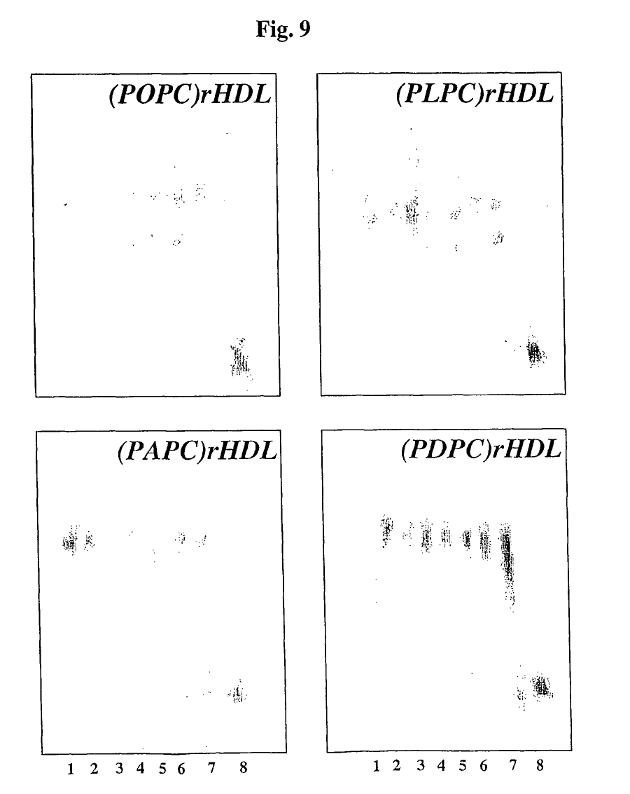

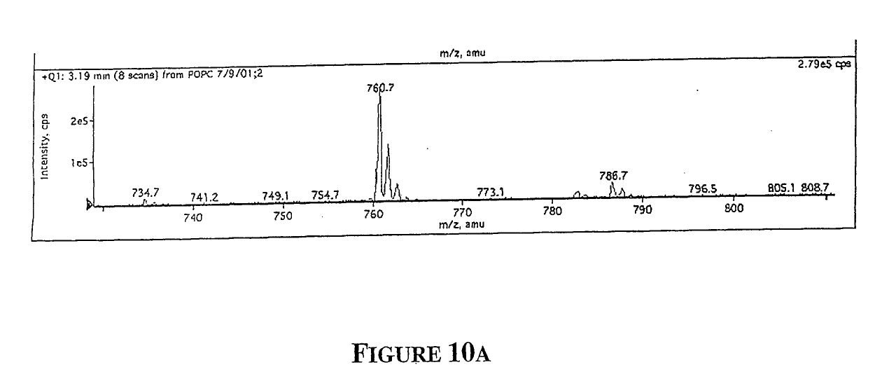

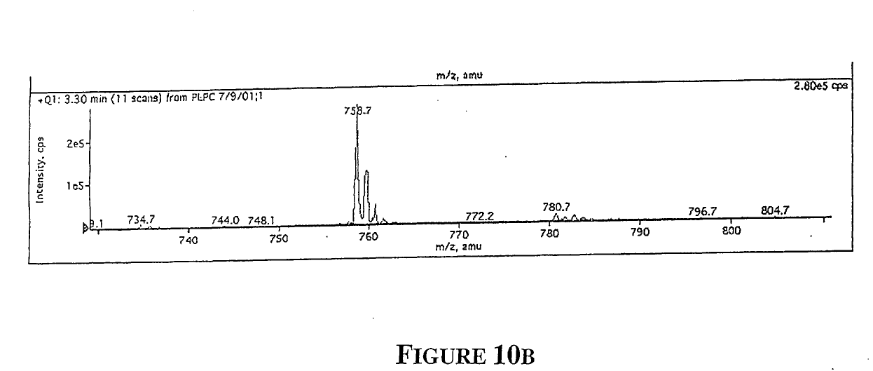

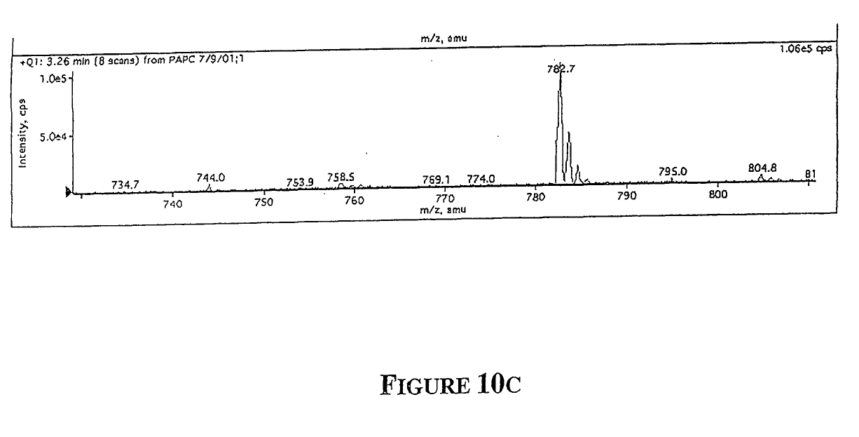

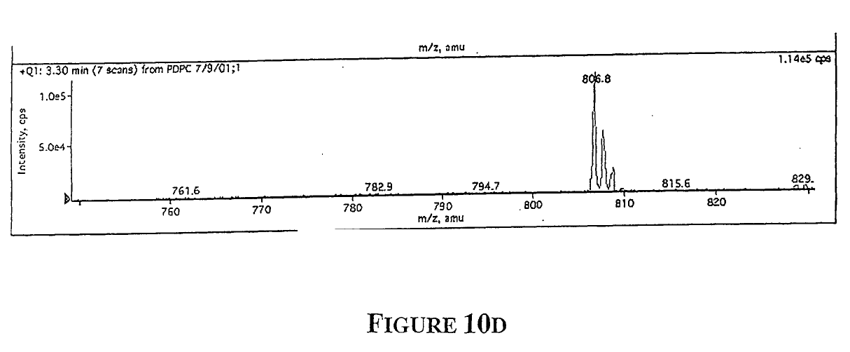

Earlier work from this laboratory has shown that apoA-I dissociates from rHDL during incubation with CETP and triglyceride-rich particles such as VLDL or Intralipid® (4). To determine how phospholipid composition affects this process the rHDL preparations were incubated with CETP and the appropriate microemulsion for 0-24 h. The incubation mixtures were then subjecting to non-denaturing gradient gel electrophoresis and the dissociation of apoA-I was assessed by immunoblotting (FIG. 9). The rHDL that were maintained at 4° C. or incubated at 37° C. for 24 h in the absence of CETP are shown in Tracks 1 and 2, respectively. Tracks 3-7 show the rHDL after incubation with CETP and the microemulsion for 1, 3, 6, 12 and 24 h. Lipid-free apo-I is shown in Track 8.

ApoA-I did not dissociate from (POPC)rHDL. In the case of (PLPC)rHDL, dissociated apoA-I was apparent after 24 h of incubation. ApoA-I dissociated from the (PAPC)rHDL and (PDPC)rHDL after 12 and 6 h of incubation, respectively.

Given that the (POPC)rHDL, (PLPC)rHDL, (PAPC)rHDL and (PDPC)rHDL were comparable in terms of size and had similar lipid/apoA-I molar ratios, it was somewhat surprising to find that there were major differences in their structure. The results in Table I show that the surface charge of (PAPC)rHDL and (PDPC)rHDL is more negative than that of either (POPC)rHDL or (PLPC)rHDL. This could be due either to variations in the organization of apoA-I on the rHDL surface, or to changes in the orientation of the rHDL phospholipid headgroups. As the TMA-DPH polarization data in FIG. 2 showed that the phospholipid headgroup packing order was similar in all of the rHDL preparations, the latter possibility was unlikely.

There was, by contrast, a high probability that the differences in rHDL surface charge reflect variations in the organization of apoA-I on the particle surface. The results in Table I show that the rHDL apoA-I Trp residues become increasingly exposed to the aqueous environment as the length and unsaturation of their phospholipid sn-2 acyl chains increases. This could be a consequence of the more bulky, unsaturated phospholipid acyl chains of PAPC and PDPC occupying larger areas and progressively excluding the apoA-I α-helices from the particle surface. The finding that the phospholipid acyl chain packing order becomes more disordered with increasing length and unsaturation of the rHDL phospholipid acyl chains (FIG. 2) also raised the possibility that apoA-I may be excluded from the rHDL surface by increased partitioning of CE from the core into the particle surface. Both of these possibilities could result in the destabilization of apoA-I. Evidence that this is the case comes from FIG. 5, which shows that the rate of unfolding of apoA-I increases with increasing length and unsaturation of the rHDL phospholipid sn-2 acyl chains.

Surface plasmon resonance was used to identify which regions of apoA-I varied according to rHDL phospholipid sn-2 acyl chain length and unsaturation. This was achieved by determining the ka for the binding of the rHDL preparations to a series of well defined, epitope-specific apoA-I-specific-monoclonal antibodies. As judged by the results in FIG. 3, the exposure of all epitopes examined along the entire length of the apoA-I molecule had altered affinities for their defining antibodies as a result of their association to the different rHDL preparations.

The finding that phospholipid composition regulates rHDL surface charge raised the possibility that ionic interactions of rHDL with plasma factors such as CETP may also be affected. As interactions of this type are fundamentally important for HDL remodeling, this prospect was investigated further. This was achieved by incubating the rHDL preparations with CETP and a phospholipid/triolein microemulsion. To ensure that the phospholipid composition of the rHDL did not change during the incubations, the phospholipid composition of the microemulsions and the rHDL were identical. The results of these experiments showed that CETP-mediated transfers of core lipids between rHDL and microemulsions increased as the length and unsaturation of the rHDL sn-2 phospholipid acyl chains increased (FIG. 6).

The results in Table II show that most of the spherical rHDL CE are generated by the LCAT-mediated hydrolysis of discoidal rHDL phospholipid sn-2 acyl chains. This raises the possibility that the differences in the CE and TG transfers in FIG. 6 reflect variations in rHDL CE, rather than phospholipid, composition. However, as the results in FIG. 6 show a clear reciprocal relationship between the CE and TG transfers, and the transfer of triolein from the microemulsions to rHDL is unlikely to be influenced by rHDL CE content, it follows that the phospholipids in the rHDL (or the microemulsions) are probably responsible for the different rates of transfer of both CE and TG.

This project provided a unique opportunity to study the relationship between all of the processes that constitute the CETP-mediated remodeling of rHDL. This includes depletion of rHDL core lipids, the reduction in rHDL particle size and the dissociation of apoA-I. FIG. 8 shows that CETP converts (PAPC)rHDL and (PDPC)rHDL into small particles more rapidly than either (POPC)rHDL or (PLPC)rHDL. Similarly, (PAPC)rHDL and (PDPC)rHDL are depleted of core lipids more rapidly than either (POPC)rHDL or (PLPC)rHDL (FIG. 7). Finally, apoA-I dissociates more readily from (PAPC)rHDL and (PDPC)rHDL than from (PLPC)rHDL (FIG. 9). When taken together these results show a high degree of cooperativity between all of the processes that constitute the CETP-mediated remodeling of rHDL.

The finding that CETP did not mediate the dissociation of apoA-I from (POPC)rHDL indicates that core lipid depletion must exceed a threshold level before apoA-I can dissociate from rHDL. This result also suggests that the stability of apoA-I in (POPC)rHDL is enhanced. The results in Table III and FIG. 5, which show that the apoA-I in (POPC)rHDL has a higher free energy of unfolding, and unfolds at a slower rate than the apoA-I in the other rHDL preparations, are consistent with this notion. While the exact reason for the increased stability of apoA-I in the (POPC)rHDL is unclear, it could be related to the more ordered packing of the phospholipid acyl chains in these particles enabling the apoA-I α-helices to partition more readily into the particle surface.

The finding that phospholipid composition regulates the dissociation of apoA-I from rHDL is of particular interest. The apoA-I that dissociates from HDL has at least three fates (i) it may be converted into new HDL particles by accepting cholesterol and phospholipids from cells in the first step in reverse cholesterol transport (6), (ii) it may be reincorporated directly into mature HDL that are increasing in size as a result of the action of LCAT (31) or (iii) it may be removed from the circulation directly via the kidneys (32). The present results show for the first time that HDL phospholipid composition can regulate processes (i) and (ii). The results also suggest that HDL phospholipid composition may be crucial for maintaining, and possibly increasing, HDL levels. Given that HDL phospholipid composition varies according to dietary fat intake (33), the physiological importance of these findings is high.

This study shows that phospholipids have a major influence on the structure of rHDL and their remodeling by CETP. The observation that phospholipids also regulate the dissociation of apoA-I from rHDL highlights their potential importance in several aspects of HDL metabolism, such as regulating plasma HDL levels and the initial step of reverse cholesterol transport.

The capacity to provide spherical rHDL with defined phospholipid compositions has potentially important practical ramification. Firstly in providing a tool that can be used to further elucidate the role of HDL particles and other lipoprotein particles and secondly in providing for greater control over the quality of lipoprotein particles for therapeutic purposes, as well as tailoring the particle to have particular properties.

REFERENCES

1. Subbaiah et al., (1992) Biochim. Biophys. Acta. 1128, 83-92.

2. Subbaiah and Monshizadegan. (1988) Biochim. Biophys. Acta. 963, 445-455.

3. Davidson et al., (1994) J. Biol. Chem. 269, 8959-8965.

4. Rye et al., (1995) J. Biol. Chem. 270, 189-196.

5. Liang et al., (1994) J. Lipid Res. 35, 1187-1199.

6. Castro and Fielding (1988) Biochemistry. 27, 25-29.

7. Rye and Barter (1994) J. Biol. Chem. 269, 10298-10303.

8. Osborne (1986) Methods Enzymol. 128, 213-222.

9. Rye (1990) Biochim. Biophys. Acta. 1042, 227-236.

10. Weisweiler (1987) Clin. Chim. Acta. 169, 249-254.

11. Rye et al., (1996) J. Biol. Chem. 271, 4243-4250.

12. Piran and Morin (1979) J. Lipid. Res. 20, 1040-1043.

13. Burstein et al., (1970) J. Lipid. Res. 11, 583-595.

14. Tollefson et al., (1988) Am. J. Physiol. 255, E894-E902.

15. Matz and Jonas (1982) J. Biol. Chem. 257, 4535-4540.

16. Parks and Gebre (1997) J. Lipid Res. 38, 266-275.

17. Martins et al., (1989) Biochim. Biophys. Acta. 1005, 217-224.

18. Sattler et al., (1994) Methods Enzymol. 233, 469-489.

19. Curtiss et al., (2000) Biochemistry. 39, 5712-5721.

20. Sparks and Phillips (1992) J. Lipid Res. 33, 123-130.

21. Rainwater et al., (1992) J. Lipid Res. 33, 1876-1881.

22. Lowry et al., (1951) J. Biol. Chem. 193, 265-275.

23. Staros (1982) Biochemistry. 21, 3950-3955.

24. Rye and Duong (2000) J. Lipid Res. 41, 1640-1650.

25. Shen et al., (1977) Proc. Natl. Acad. Sci.(USA). 74, 837-841.

26. Baker et al., (2000) J. Lipid Res. 41, 1261-1267.

27. Fournier et al., (1997) Arterioscler. Thromb. Vasc. Biol. 17, 2685-2691.

28. Tollefson et al., (1988) J. Lipid Res. 29, 1593-1602.

29. Kawano et al., (2000) J. Biol. Chem. 275, 29477-29481.

30. Schaefer et al., (1982) J. Lipid Res. 23, 1259-1273.

31. Liang et al., (1996) J. Lipid Res. 37, 1962-1970.

32. Horowitz et al., (1992) In: High Density Lipoproteins and Atherosclerosis III. Miller and Tall, eds. Amsterdam; London; New York; Tokyo: Excerpta Medica, 215-222.

33. Hodge et al., (1993) Lipids. 28, 525-531.

34. Persson et al., (1999) Biotech Bioeng. 65:371-381

35. Persson et al., (1998) J Chromatogr B Biomed Sci Appl. 711:97-109

36. Kiss et al., (1998) Protein Expr Purif. 12:353-360

37. Xiong (1997) J Chromatogr B Biomed Sci Appl 702:216-220

38. Lopez et al., (1994) Eur J Biochem. 225:1141-1150

39. Sigalov et al., (1991) J Chromatogr. 537:464-468

40. Forstner et al., (1999) Protein Expr Purif. 17:267-272

41. Pillot et al., (1996) Protein Expr Purif. 7:407-414

42. Yamamoto et al., (1998) Am J Vet Res. 59:667-672

43. Wang et al., (1996) Biochim Biophys Acta. 1302:224-230

1. A method of forming spheroidal reconstituted HDL particles, including the step of forming a disc shaped HDL particle with a defined phospholipid composition, and the step of making the disc shaped HDL particles spheroidal including multiple stepwise addition of unesterified cholesterol carried in a solvent, followed by addition of a cholesterol esterifying agent capable of esterifying the unesterified cholesterol using acyl constituents of the phospholipids.

2. A method of forming spheroidal reconstituted HDL particles as in claim 1 wherein the cholesterol esterifying agent is the enzyme LCAT.

3. A method of forming spheroidal reconstituted HDL particles as in claim 2 wherein the solvent used for carrying the unesterified cholesterol is a polar solvent that is miscible with water.

4. A method of forming spheroidal reconstituted HDL particles as in claim 3 wherein the solvent is ethanol.

5. A method of forming spheroidal reconstituted HDL particles as in claim 3 wherein the method include the additional step of introducing an adsorbing agent that can selectively adsorb the lyso phospholipid that forms as a result of the activity of LCAT.

6. A method of forming spheroidal reconstituted HDL particles as in claim 5 wherein the adsorbing agent is a protein.

7. A method of forming spheroidal reconstituted HDL particles as in claim 6 wherein the protein is an albumin.

8. A method of forming spheroidal reconstituted HDL particles as in claim 4 wherein an LCAT activator is additionally added.

9. A method of forming spheroidal reconstituted HDL particles as in claim 8 wherein the LCAT activator is β-mercaptoethanol (βME).

10. A method of forming spheroidal reconstituted HDL particles as in claim 8 wherein the lyso phospholipid adsorbing agent, reducing agent, esterification agent, and unesterified cholesterol are added to disc shaped HDL particles in the sequence listed.

11. A method of forming spheroidal reconstituted HDL particles as in claim 1 wherein the molar ratio of esterified cholestrol to apoA-I protein is between 10:1 to 40:1.

12. A method of forming spheroidal reconstituted HDL particles as in claim 1 wherein the molar ratio of esterified cholestrol to apoA-I protein is between 15:1 to 30:1.

13. A method of forming spheroidal reconstituted HDL particles as in claim 1 wherein the molar ratio of esterified cholestrol to apoA-I protein is between 15:1 to 20:1.

14. A method of forming spheroidal reconstituted HDL particles as in claim 1 wherein the discoidal rHDL has a ratio of phospholipids to apoprotein A-I of about 100:1.

15. A method of forming spheroidal reconstituted HDL particles as in claim 1 wherein head groups of the phospholipids constituting the particle are selected from the group consisting of phosphatidyl serine, phosphatidylinositol, phosphatidyl ethanolamine and sphingomyelin.

16. A method of forming spheroidal reconstituted HDL particles as in claim 1 wherein head groups of the phospholipids constituting the particle are selected from the group consisting of phosphatidyl glycerol, phosphatidyl serine, phosphatidyl inositol, phsophatidyl ethanolamine, cerebroside or a ganglioside.

17. A method of forming spheroidal reconstituted HDL particles as in claim 1 wherein fatty acid substituents of phospholipids contituting the HDL particle are selected from the group consisting of 1-palmitoyl-2-oleoyl-, 1-palmitoyl-2-linoleoyl-, 1-palmitoly-2-arachadonyl-, 1-palmitoyl-2-docosahexanoyl.

18. A method of forming spheroidal reconstituted HDL particles as in claim 1 wherein fatty acid substituents of phospholiids contituting the HDL particle are selected from the group consisting of fatty acyl groups having acyl chains of about 12 to about 18 carbon atoms.

19. A method of forming spheroidal reconstituted HDL particles as in claim 1 wherein the particle is formed to have phospholipid of wholly one type.

20. A method of forming spheroidal reconstituted HDL particles as in claim 1 wherein the particle is formed to have solely PLPC as the phosholipid content.

21. A method of forming spheroidal reconstituted HDL particles as in claim 1 wherein the particle is formed to have two or more phosholipids.

22. A method of forming spheroidal reconstituted HDL particles as in claim 21 wherein the defined proportions are determined at the stage of forming the discoidal rHDL so that thephospholipid content of the discoidal HDL particle is the defined proportion.

23. A method of forming spheroidal reconstituted HDL particles as in claim 21 including the step of making spheroidal HDL with a phospholipid composition containing only one type, and one or more further phospholipids are added to a reaction mixture in the form of a vesicle, and the vesicle might be made wholly of one phospholipid and an phospholipid transfer enzyme is added to transfer enzymes from the vesicle to the HDL particle.

24. A method of forming spheroidal reconstituted HDL particles as in claim 23 wherein the phospholipid transfer enzyme is PLPT.

25. A preparation of spheroidal reconstituted HDL particles with a defined phospholipid composition.

26. A preparation of spheroidal reconstituted HDL particles as in claim 25 formed from disc shaped HDL particles with a defined phospholipid composition by the multiple stepwise addition of unesterified cholesterol carried in a solvent, followed by addition of a cholesterol esterifying agent capable of esterifying the unesterified cholesterol using acyl constituents of the phospholipids.

27. A preparation of spheroidal reconstituted HDL particles as in claim 25 wherein the cholesterol esterifying agent is the enzyme LCAT.

28. A preparation of spheroidal reconstituted HDL particles as in claim 27 wherein the method include the additional step of introducing an adsorbing agent that can selectively adsorb the lyso phospholipid that forms as a result of the activity of LCAT.

29. A preparation of spheroidal reconstituted HDL particles as in claim 28 wherein an LCAT activator is additionally added.

30. A preparation of spheroidal reconstituted HDL particles as in claim 25 wherein the molar ratio of esterified cholestrol to apoA-I protein is between 10:1 to 40:1.

31. A preparation of spheroidal reconstituted HDL particles as in claim 25 wherein the molar ratio of esterified cholestrol to apoA-I protein is between 15:1 to 30:1.

32. A preparation of spheroidal reconstituted HDL particles as in claim 25 wherein the molar ratio of esterified cholestrol to apoA-I protein is between 15:1 to 20:1.

33. A preparation of spheroidal reconstituted HDL particles as in claim 25 wherein the discoidal rHDL has a ratio of phospholipids to apoprotein A-I of about 100:1.

34. A preparation of spheroidal reconstituted HDL particles as in claim 25 wherein head groups of the phospholipids constituting the particle are selected from the group consisting of phosphatidyl serine, phosphatidylinositol, phosphatidyl ethanolamine and sphingomyelin.

35. A preparation of spheroidal reconstituted HDL particles as in claim 25 wherein head groups of the phospholipids constituting the particle are selected from the group consisting of phosphatidyl glycerol, phosphatidyl serine, phosphatidyl inositol, phsophatidyl ethanolamine, cerebroside or a ganglioside.

36. A preparation of spheroidal reconstituted HDL particles as in claim 25 wherein fatty acid substituents of phospholipids contituting the HDL particle are selected from the group consisting of 1-palmitoyl-2-oleoyl-, 1-palmitoyl-2-linoleoyl-, 1-palmitoly-2-arachadonyl-, 1-palmitoyl-2-docosahexanoyl.

37. A preparation of spheroidal reconstituted HDL particles as in claim 25 wherein fatty acid substituents of phospholipids contituting the HDL particle are selected from the group consisting of fatty acyl groups having acyl chains of about 12 to about 18 carbon atoms.

38. A preparation of spheroidal reconstituted HDL particles as in claim 25 wherein the particle has a phospholipid composition of wholly one type.

39. A preparation of spheroidal reconstituted HDL particles as in claim 25 wherein the particle is formed to have phosholipid solely in the form of PLPC.

40. A preparation of spheroidal reconstituted HDL particles as in claim 25 wherein the particle is formed to have two or more phospholipids.
